(19) United States
(12) Patent Application Publication
(10) Pub. No.: US 2008/0268050 A1
(43) Pub. Date: Oct. 30, 2008

(54) SPRAYABLE TOPICAL SKIN BARRIERS

(76) Inventors: Donald L. Gerrish, Mankato, MN (US); Charles E. Boyer, Elysian, MN (US)

Correspondence Address: COLOPLAST CORP.;I.P. DEPARTMENT, U.S. OPERATIONS, P.O. BOX 580800, MINNEAPOLIS, MN, 55456-0800, US

(21) Appl. No.: 11/742,066

(22) Filed: Apr. 30, 2007

(51) Int. Cl. A61K 9/00; A61K 31/24; A61K 31/355; A61P 17/00; A61K 31/415

(52) U.S. Cl. 424/484; 514/396; 514/458; 514/535

(57) ABSTRACT

Sprayable topical skin barriers for protecting and promoting healing of skin, and for providing comfort to a patient, comprise (i) a semi-solid hydrocarbon in a range by weight from about 25.0% to about 90.0%, (ii) a water-absorbing compound having a particle size range of about 0.5 microns to 20.0 microns, in a range by weight from about 5.0% to about 75.0%, and (iii) a solvent in a range by weight from about 10.0% to about 70.0%. The sprayable topical skin barriers have properties of effective adhesion to skin, protection from moisture and waste, transparency, sprayability, and smoothness to touch.

### FIELD OF THE INVENTION

The present invention relates generally to topical skin barriers. The invention relates specifically to sprayable topical skin barriers for protecting and promoting healing of patients' skin, and for providing comfort to patients.

### BACKGROUND OF THE INVENTION

Topical skin barrier compositions, hereinafter referred to as “topical skin barriers”, are known in the medical arts. Topical skin barriers have been used, inter alia, for the treatment of bedridden patients' skin where irritation from moisture, urine, diarrhea, feces, enzymatic drainage, exudate, dust, dirt, and the like (hereinafter, collectively, “moisture and waste”) is problematic, painful, and unfortunately commonplace. Patients' skin, regardless of being intact or non-intact, ideally needs to be protected from moisture and waste to prevent skin breakdown, promote healing, and provide comfort.

Ultimately it is the role of topical skin barriers to protect skin exposed to moisture and waste, since enzymes present in waste can quickly lead to skin breakdown. While any topical skin barrier that protects skin from exposure to moisture and waste, acting as a barrier therefrom, may be beneficial, an ability to remain adhered to both intact and non-intact skin is obviously critical to satisfactory performance. Furthermore, known topical skin barrier compositions are often too thick to be dispensed as a spray due to particle sizes of their water-absorbing compounds which precipitate out when solvent is added in attempts to make them sprayable.

Therefore, there has existed a long-felt need for sprayable topical skin barriers for general skin care which are not limited in application to a specific area of a patient's body. Such products could also optionally include antifungal and pain relief agents. The sprayable topical skin barriers would need to exhibit effective adhesion to skin and provide a good barrier in an environment of moisture and waste. Moreover, it would be desirable for such products to optionally have “detectable transparency”, thereby rendering them detectable to an observer while being substantially transparent to permit visual observation of patients' skin thereunder.

### SUMMARY OF THE INVENTION

In accordance with basic aspects of the present invention, sprayable topical skin barriers for protecting and promoting healing of skin, and for providing comfort to a patient, comprise (i) a semi-solid hydrocarbon in a range by weight from about 25.0% to about 90.0%, (ii) a water-absorbing compound having a particle size range of about 0.5 microns to 20.0 microns, in a range by weight from about 5.0% to about 75.0%, and (iii) a solvent in a range by weight from about 10.0% to about 70.0%. The sprayable topical skin barriers have properties of effective adhesion to skin, protection from moisture and waste, transparency, sprayability, and smoothness to touch.

### DETAILED DESCRIPTION OF THE INVENTION

As used here throughout, the term “water-absorbing compound” is intended to include any suitable compound such as, for example, (i) cellulose gum, whether identified as water-absorbing or otherwise, (ii) carboxymethylcellulose, commonly referred to as “CMC” and which is commercially available, for example, as BLANOSE® brand water soluble polymer from Hercules Incorporated of Wilmington, Del., (iii) karaya gum, and even (iv) specific brands of superabsorbent polymers such as WATER LOCK® G-430 and WATER LOCK® A-240, each being commercially available from Grain Processing Corporation of Muscatine, Iowa.

Several alternative compositions have been formulated as sprayable topical skin barriers of the present invention. These are presented as the following Examples 1-5 (with ranges expressed in percentages by weight):

| | Example 1 | Example 2 |
|---|---|---|
| Semi-solid hydrocarbon | 25.00–95.00 | 25.00–95.00 |
| Water-absorbing compound | 05.00–75.00 | 05.00–75.00 |
| Solvent | 10.00–70.00 | 10.00–70.00 |
| Skin conditioning agent | — | 00.10–10.00 |
| | Example 3 | Example 4 |
| Semi-solid hydrocarbon | 25.00–95.00 | 25.00–95.00 |
| Water-absorbing compound | 05.00–75.00 | 05.00–75.00 |
| Solvent | 10.00–70.00 | 10.00–70.00 |
| Aesthetic agent | 00.10–20.00 | — |
| Antifungal agent | — | 00.50–02.50 |

| | Example 5 |
|---|---|
| Semi-solid hydrocarbon | 25.00–95.00 |
| Water-absorbing compound | 05.00–75.00 |
| Solvent | 10.00–70.00 |
| Pain relief agent | 00.25–20.00 |

The present invention, as illustrated by the foregoing examples, may be dispensed for use from a spray container due to (i) presence of the solvent which acts on the formulations to enable them to readily pass through mechanical components of spray containers and (ii) perhaps more importantly, a reduction in particle size and hence “grittiness” of the water-absorbing compound relative to known skin barrier formulations that are dispensed by, e.g., squeezing them out from a tube. Particle sizes of water-absorbing compounds in the alternative formulations of the present invention are reduced to about 0.5-20 microns to make them sprayable and smooth to the touch (i.e., to reduce grittiness). Equipment as commercially available from, e.g., Microfluidics Corp. of Newton, Mass., is utilized to reduce the particle size or grittiness of the water-absorbing compound.

Preferably, in each example: the semi-solid hydrocarbon is about 40%; the water-absorbing compound is about 13%; and the solvent is about 47%. The solvent could be low molecular weight hydrocarbons, volatile silicones, or volatile fluorocarbons (e.g., isooctane, hexamethyldisiloxane, and cyclomethicone). Also preferably, in each example, a ratio of semi-solid hydrocarbon (e.g., petrolatum) to water-absorbing compound (e.g., CMC) is about 3:1.

Also preferably: in Example 2 the skin conditioning agent (e.g., tocopherol or tocopheryl acetate) is about 0.5%; in Example 3 the aesthetic agent (for, inter alia, detectability or “detectable transparency”; e.g., zinc oxide and titanium dioxide) is about 1.0%; in Example 4 the antifungal agent is about 2.0%; and Example 5 the pain relief agent is about 1.0%.

It is to be understood in Example 2 that the skin conditioning agent may also be, either alone or in combination, a skin nutrient.

It is to be understood in Example 3 that the aesthetic agent is intended to provide attributes in the formulation, either alone or in combination with each other, of (i) detectability or “detectable transparency” and (ii) an appealing color which may be an appealing or subjectively aesthetic quality. As used here throughout, the term “detectable transparency” is intended to include a characteristic of being detectable to an observer while being substantially transparent to permit visual observation of skin thereunder. In this regard, those of skill in the art will recognize that known topical skin barriers are often substantially opaque after application to skin which therefore does not allow the condition of the skin to be visually assessed. This can lead to a perceived need to aggressively remove selected portions of the topical skin barrier to visually inspect the skin thereunder. Such aggressive removal, in turn, can lead to further injury to the skin.

As presented in Example 4, further discoveries have been made relative to development of an antifungal property in the sprayable topical skin barriers of the present invention. The addition of a suitable antifungal agent, such as, for example, miconazole nitrate or clotrimazole, is desirable in some instances because the aforedescribed skin maladies are often susceptible to fungal infections. In this regard, it has been further discovered that such addition of a suitable antifungal agent gives an unexpected, additional result of an enhanced moisture and waste barrier property in a given formulation of the present invention. In a preferred, exemplary embodiment, utilization of miconazole nitrate in a range by weight from about 1.5% to about 2.5% alleviates a need for an aesthetic agent which may as desired lighten a coloration appearance of a given formulation. As an alternative clotrimazole may be substituted for miconazole nitrate, in a range by weight from about 0.5% to about 2.0%. Specifically, it has been found that the addition of miconazole nitrate or clotrimazole in such proportions inherently provides an appealing subjectively aesthetic quality along with detectable transparency.

In development of further preferred or exemplary embodiments of the present invention not stated above, it was recognized that utilization of an antifungal agent often occurs in an unpleasant odor-producing fungal environment. Therefore, it may be desirable to add an odor control agent to a given formulation of the invention, in a range by weight from about 0.1% to about 10.0%. A suitable odor control agent could be virtually any compatible, commercially available fragrance or deodorizer such as, for example, ORDENONE® brand deodorizer from Belle-Aire Fragrances, Inc., of Mundelein, Ill.

As presented in Example 5, it has been discovered that the sprayable topical skin barriers of the present invention may advantageously include a pain relief agent for patient comfort. The addition of a suitable pain relief agent, such as, for example, dibucaine, lidocaine, benzocaine, betamethasone, or tetracaine, in a range by weight from about 0.25% to about 20.0% depending upon the particular agent chosen as recognized by those in the medical or pharmaceutical arts, is desirable in some instances because the aforedescribed skin maladies may be painful.

It is to be appreciated from the foregoing disclosure that the present invention satisfies the long-felt need for a sprayable topical skin barrier for general skin care which (i) is not limited in application to a specific area of a patient's body, (ii) optionally includes antifungal and pain relief agents, (iii) adheres well to skin and provides a good barrier in an environment of moisture and waste, and (iv) has detectable transparency. The present invention, therefore, may be further characterized, for example, as performing at least as satisfactorily as the various CRITIC-AID® brand topical skin barriers from Coloplast A/S of Denmark.

It is to be recognized by those skilled in the medical or pharmaceutical arts that the sprayable topical skin barriers of the present invention may be properly characterized as being anhydrous, which is known to be hostile to microbes. Such an environment provided by the formulations of the present invention would, therefore, advantageously afford a low risk of microbial contamination. Consequently, it is to be particularly noted, the various alternative compositions of the present invention do not require the addition of a preservative. Accordingly, the present invention may be properly characterized as being preservative-free.

It is also to be recognized that although the aforedescribed example embodiments of the present invention are sprayable, they could just as well be applied conventionally by hand such as, for example, squeezed out from a tube. Furthermore, the aforedescribed reduction of particle sizes in the water-absorbing compounds of the present invention has been found by patients and caregivers to feel better in use as compared to conventional non-sprayable skin barriers.

While the present invention has been particularly shown and described with reference to the accompanying specification, it will be understood however that other modifications thereto are of course possible; and all of which are intended to be within the true spirit and scope of the present invention. It should be appreciated that (i) components, dimensions, formulations, and other particulars of example embodiments of the invention aforedescribed may be substituted for others which are suitable for achieving desired results, (ii) various additions or subtractions may be made thereto, and (iii) formulations of the foregoing examples may also be made in combinations thereof. It is also to be understood in general that any suitable alternatives may be employed to provide the sprayable topical skin barriers of the present invention.

It is to be noted that terms used here throughout are intended to have their usual, customary, and ordinary meanings, unless another is specified. In particular, it is to be understood that the following terms include, but are not limited to, the following associated meanings: “comfort” means a feeling of relief from unpleasant physical sensations; “protecting” means covering or shielding from exposure, injury, or destruction; “promoting healing” means contributing to, or advancing, a healing process; and “effective adhesion” means being operative to provide adequate adhesion.

Lastly, of course, the choice of compositions, sizes, and strengths of various aforementioned elements of the products of the present invention are all a matter of design choice depending upon intended uses thereof.

Accordingly, these and other various changes or modifications in form and detail of the present invention may also be made therein, again without departing from the true spirit and scope of the invention as defined by the appended claims.

What is claimed is:

1. A sprayable topical skin barrier for (i) protecting, and promoting healing of, skin, and (ii) providing comfort to a patient, comprising:
in a range by weight from about 25.0% to about 90.0%, a semi-solid hydrocarbon;
in a range by weight from about 5.0% to about 75.0%, a water-absorbing compound having a particle size range of about 0.5 microns to 20.0 microns; and
in a range by weight from about 10.0% to about 70.0%, a solvent,
wherein said sprayable topical skin barrier has properties of (i) effective adhesion to skin, (ii) protection from moisture and waste, (iii) transparency, (iv) sprayability, and (v) smoothness to touch.

2. The sprayable topical skin barrier of claim 1, further comprising, in a range by weight from about 0.1% to about 10.0%, a skin conditioning agent.

3. The sprayable topical skin barrier of claim 1, further comprising, in a range by weight from about 0.1% to about 20.0%, an aesthetic agent.

4. The sprayable topical skin barrier of claim 1, further comprising, in a range by weight from about 0.5% to about 2.5%, an antifungal agent.

5. The sprayable topical skin barrier of claim 1, further comprising, in a range by weight from about 0.1% to about 10.0%, an odor control agent.

6. The sprayable topical skin barrier of claim 1, further comprising, in a range by weight from about 0.25% to about 20.0%, a pain relief agent.

7. The sprayable topical skin barrier of claim 1, wherein said semi-solid hydrocarbon is selected from the group consisting of petrolatum and white petrolatum.

8. The sprayable topical skin barrier of claim 1, wherein said water-absorbing compound is selected from the group consisting of cellulose gum, water-absorbing cellulose gum, carboxymethylcellulose, karaya gum, and a superabsorbent polymer.

9. The sprayable topical skin barrier of claim 2, wherein said skin conditioning agent is selected from the group consisting of tocopherol and tocopheryl acetate.

10. The sprayable topical skin barrier of claim 6, wherein said pain relief agent is selected from the group consisting of dibucaine, lidocaine, benzocaine, betamethasone, and tetracaine.

11. The sprayable topical skin barrier of claim 4, wherein said antifungal agent is selected from the group consisting of miconazole nitrate and clotrimazole.

12. The sprayable topical skin barrier of claim 1, further comprising an anhydrous, preservative-free composition which provides an environment that is hostile to microbes and thereby affords a low risk of microbial contamination.
